(19) United States
(12) Patent Application Publication
(10) Pub. No.: US 2023/0302181 A1
(43) Pub. Date: Sep. 28, 2023

(54) AUTOMATIC PLANT FOR THE SANITIZATION AND DISINFECTION OF SHOPPING CARTS

(71) Applicants: Franco PONTONE, Cassino (IT); Michele PONTONE, Cassino (IT)

(72) Inventors: Franco PONTONE, Cassino (IT); Michele PONTONE, Cassino (IT)

(21) Appl. No.: 18/020,633

(22) PCT Filed: Aug. 9, 2021
(86) PCT No.: PCT/IT2021/050254

§ 371 Date: Feb. 10, 2023

(30) Foreign Application Priority Data
Aug. 10, 2020 (IT) 102020000007765

(51) Int. Cl. A61L 2/24; A61L 2/22; A61L 2/10; G07F 7/06; B65G 15/30

(52) U.S. Cl. CPC A61L 2/24; A61L 2/22; A61L 2/10; G07F 7/0636; B65G 15/30; A61L 2202/123; A61L 2202/15; A61L 2202/14; A61L 2202/11; B60S 3/00

(57) ABSTRACT

The invention relates to a fully automated sanitizing system (1) for shopping trolleys and the like. It provides an automated sanitization and disinfection process, divided into four phases, which guarantees the user to pick up the perfectly sanitized trolley without contact with third parties and steps for other hands, ready for its first use. The system (1) in question consists of an automated island to be placed in the parking area of supermarket or shopping center and consists of two adjacent units, one of which is intended for the storage of used trolleys where subsequently will carry out the sanitization cycle, the other destined to shelter and store the trolleys already sanitized and ready for collection in several rows. The sampling area has been designed in compliance with the anti distances aggregation. The use of this system (1) guarantees the almost total elimination of viral and bacterial loads on the trolleys.

### TECHNICAL FIELD

The present invention relates to a totally automated plant for the sanitization of everyday and promiscuous goods, in the case in point the shopping carts, whether they are made of metal or plastic, and/or other models for warehouse use and other types used for the transport of goods, destined mainly to contain products purchased from a wholesaler or a retailer, both in small and large area of sale, to be brought to his home.

Such uses represent only one example of possible application of the uses to which the automatic sanitizing plant object of the present invention is intended.

### ALTHOUGH THERE

Exist sanitizing systems operating in different sectors, they are not the applicants, in the specific field of shopping carts, whether metal or plastic, and/or other models for warehouse use and other types used for the transport of goods, previous automatic systems specific for the sanitization of trolleys, since the cleaning works are carried out occasionally and manually, or in any case with systems involving waiting times between the removal of the trolley by the user and its sanitization, but above all, at the time of the withdrawal, the above mentioned trolleys are not yet subjected to the sanitizing process for the execution of which it is the user who has to be diligent. Therefore, the user does not find perfectly clean, nor sanitised and disinfected trolleys available.

The present invention provides, instead, an automated sanitizing and disinfecting process as better described below, which guarantees to the user, at the time of withdrawal, to hold and have at his disposal the trolley totally and perfectly sanitized, without previous contacts with third parties and passages for other hands. The object of this invention is to allow the user to grip and operate the carriage which is perfectly sanitized and ready for first use.

### DISCLOSURE OF THE INVENTION

In line with the present invention, the technical aim and the proposed solution are obtained by means of the automated sanitizing and disinfecting plant on the basis of the characteristics of the appended claims, ensuring effective cleaning and sanitization, excluding any active intervention by the user and eliminating any waiting time for the sanitizing process to be carried out.

### BRIEF DESCRIPTION OF THE DRAWINGS

The following drawings and their brief description make even more evident the advantages and the practical application field, exemplifying and not limiting, of the present invention.

FIG. 1 represents a horizontal representation showing the implant in its entirety consisting of 4 parts and/or zones, respectively identified as zone A, zone B, zone C, zone D with sequential operation corresponding to 4 phases called phase 1, phase 2, phase 3 and phase 4.

FIG. 2 represents a lateral perspective view of the area of entry of the trolleys to be sanitized and of the subsequent area destined to the sanitization (steps 1, 2 and 3).

FIG. 3 shows the sanification section of the trolleys.

FIG. 4 shows the section of exit from the sanitizing zone, and of displacement and positioning of the trolleys in storage lanes.

FIG. 5 and FIG. 6 shows the subsequent section for picking up the single carriage by the user.

FIG. 7 represents the island photovoltaic system located on the cover of the system with antenna for remote data transmission.

FIG. 8 and FIG. 9 represents the lateral elevation of the plant in its entirety.

FIG. 10 shows and specifies the electromechanical process scheme.

### DETAILED DESCRIPTION OF THE INVENTION

The invention consists of an automated island for sanitizing shopping carts located in the parking lot or, in any case, in a space in front of the entrance of the shop, supermarket or shopping center. The island consists of two adjacent units, one of which is intended for the storage of used trolleys, where the sanitizing cycle will subsequently take place, and the other one is intended for the storage and storage, on several rows, of trolleys already sanified and ready for removal. The sampling area has been studied in compliance with the anti-aggregation distances.

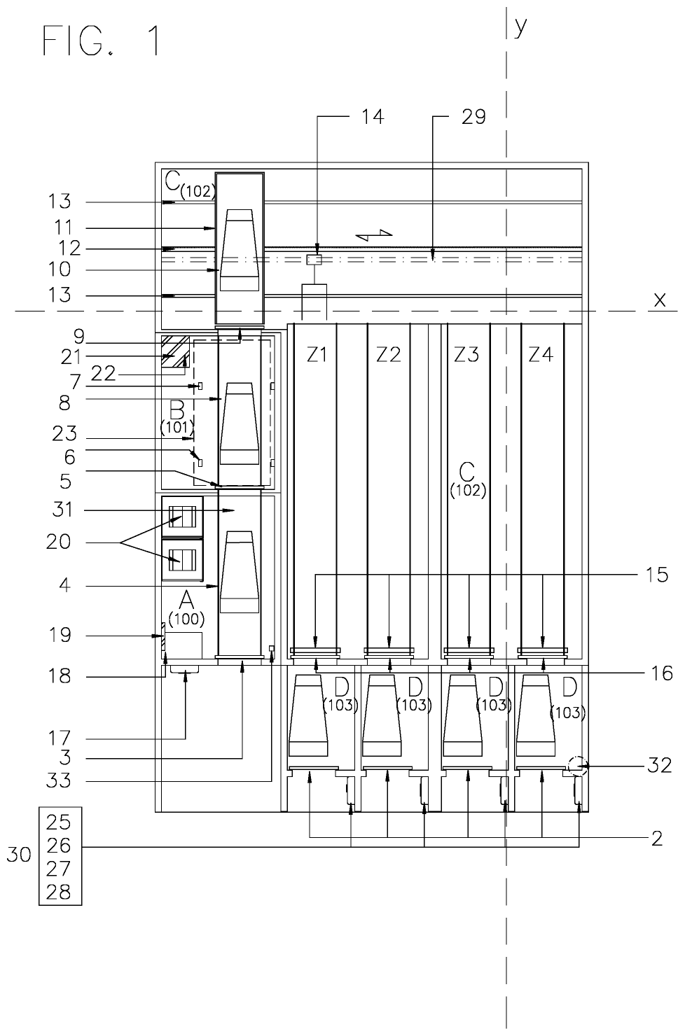

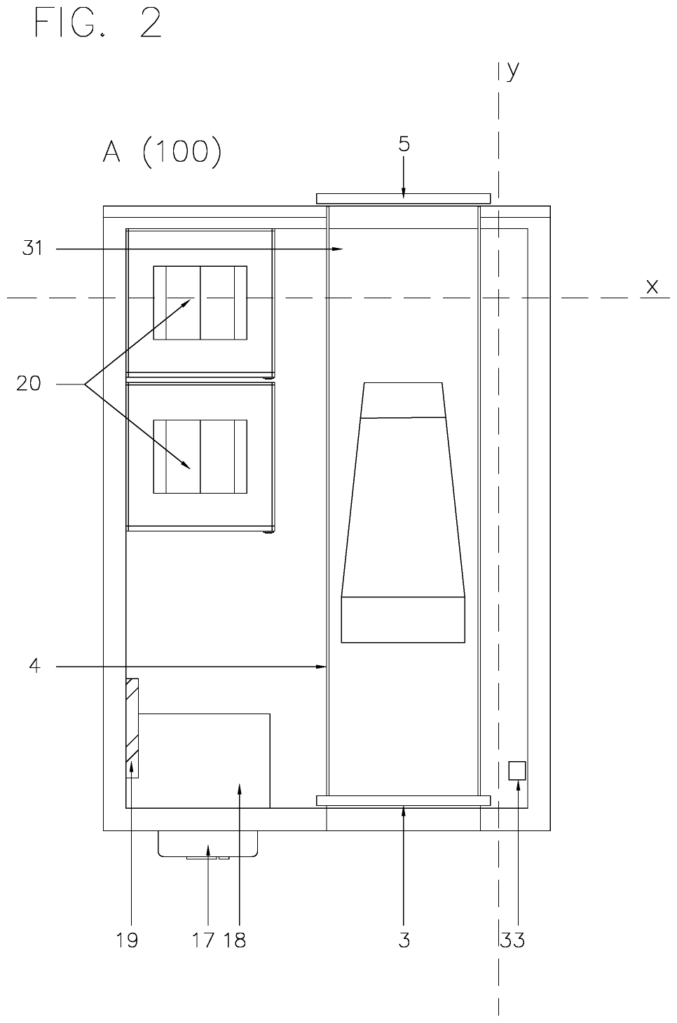

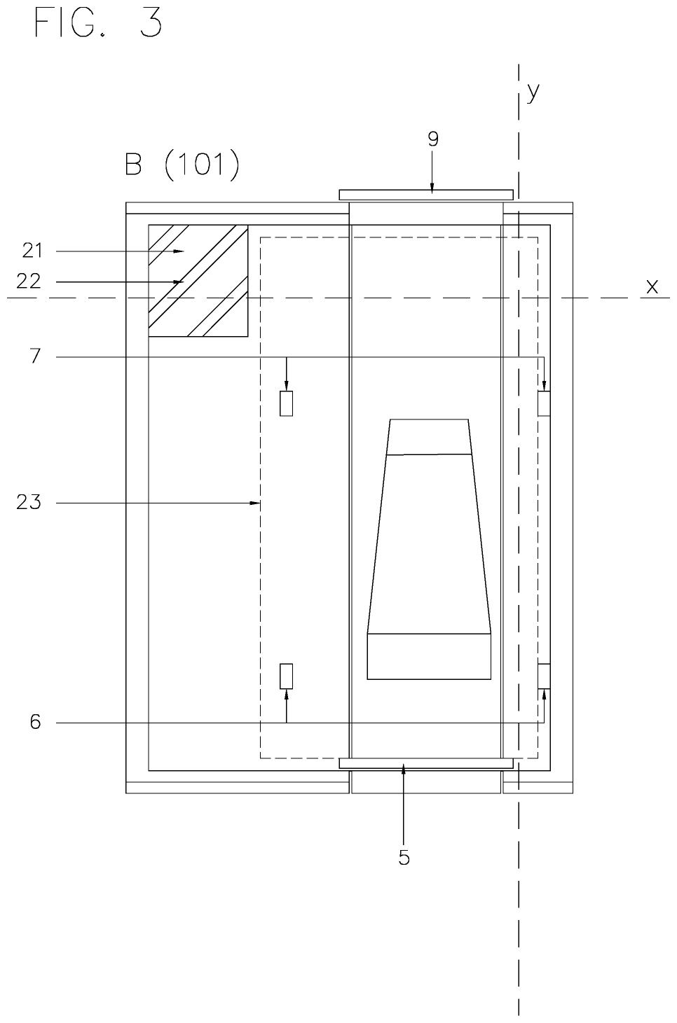

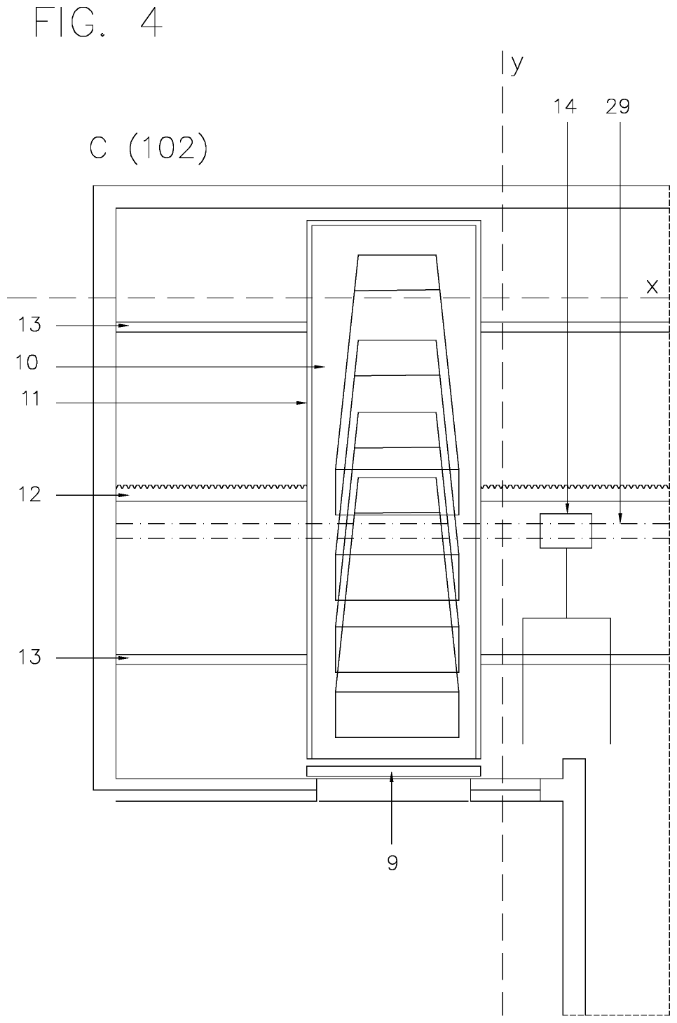

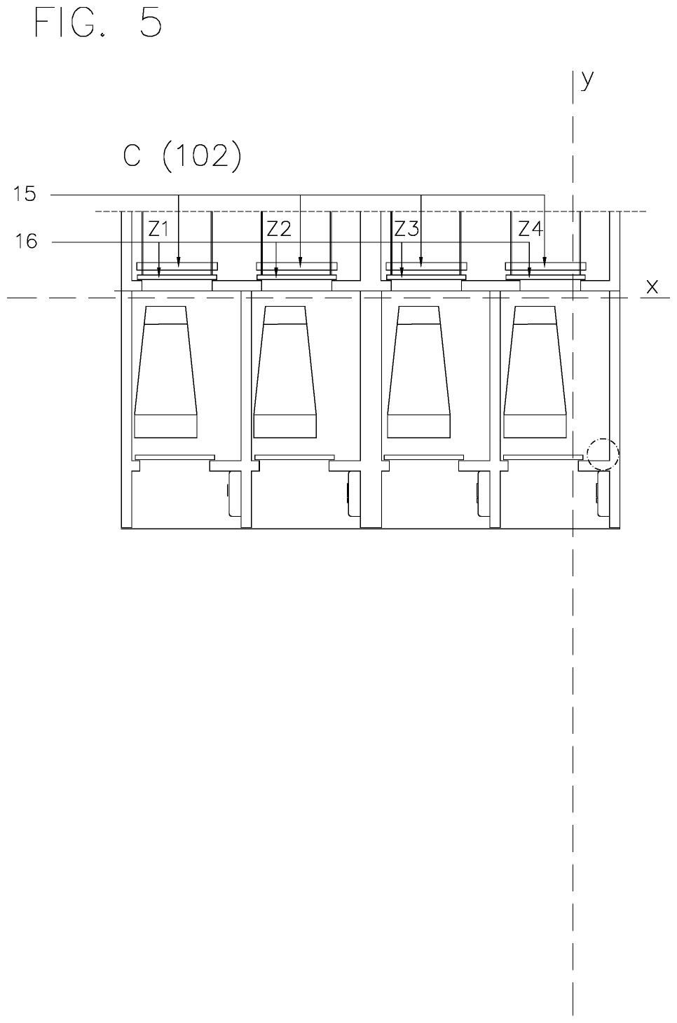

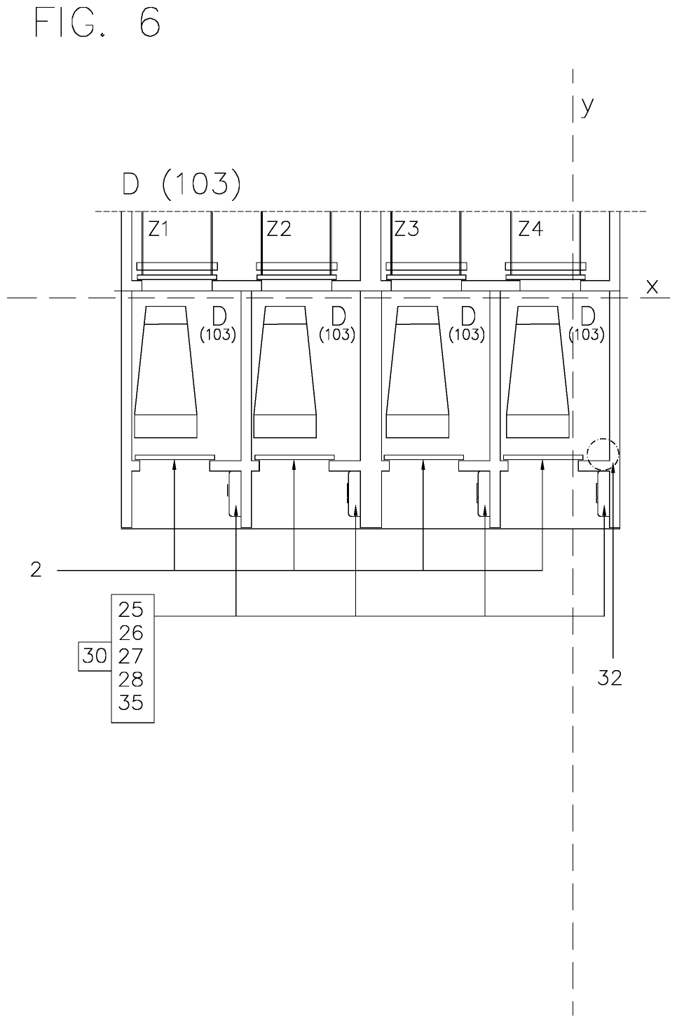

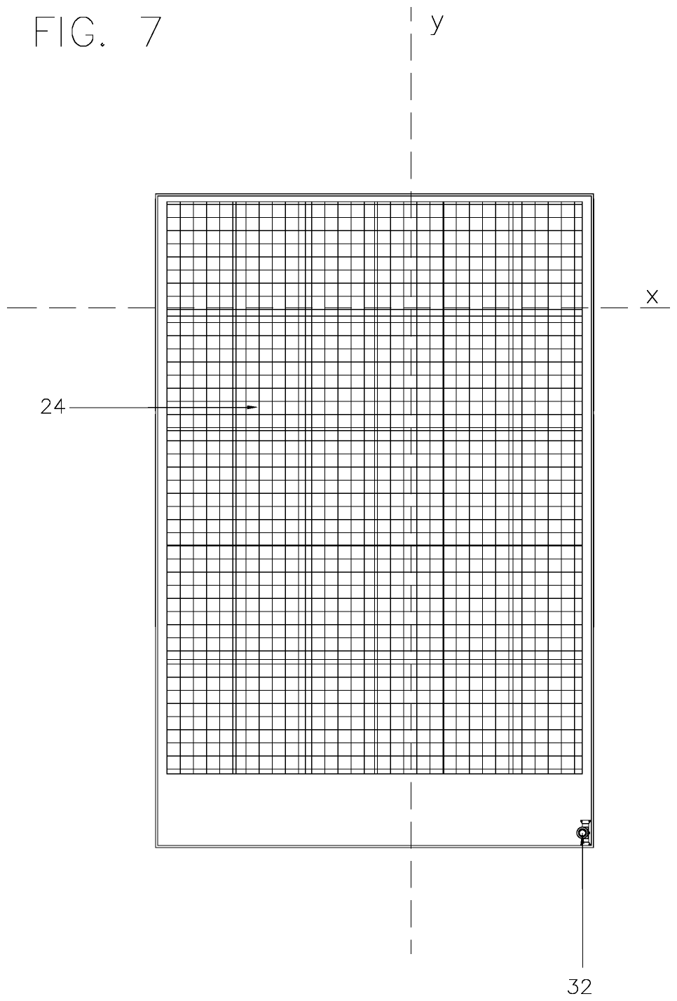

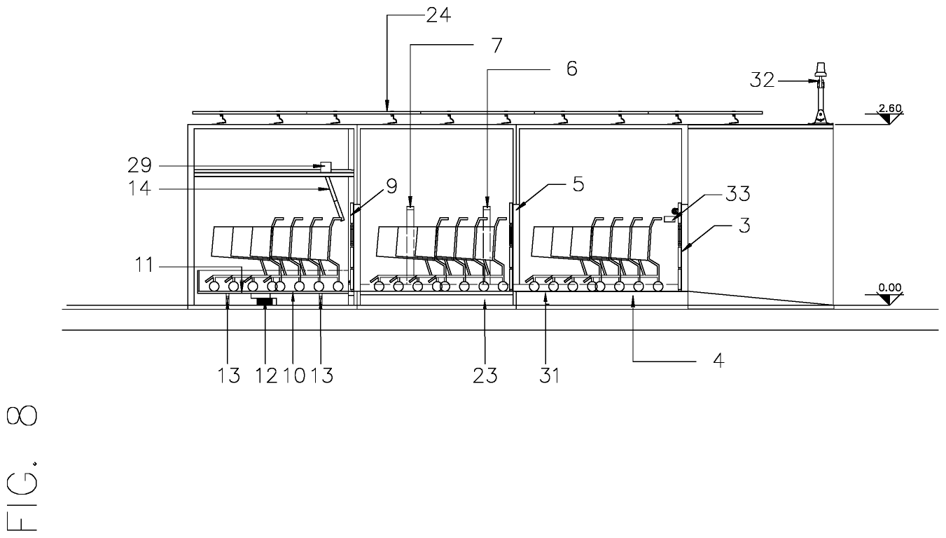

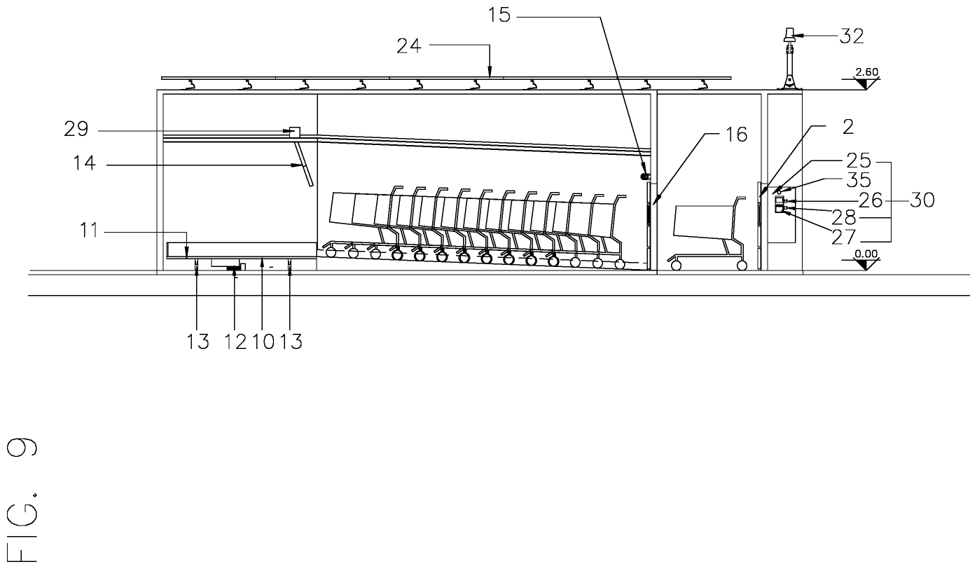

The following will be followed by an analytical description of both mechanical and electronic components and, therefore, an exposure of the plant both in the logical-functional and in the plant-realization aspect, set forth in its entirety in the general plan of FIG. 1. In the above drawing, therefore, the plant as a whole is shown, consisting of 4 parts with sequential operation, corresponding to the four steps of the sanitizing and disinfecting process thus distinct:
Step 1 corresponding to the “zone A” (FIG. 2): Loading and storage room used trolleys;
Phase 2 corresponding to the “zone B” (FIG. 3): Local sanitizing trolleys (washing and drying); phase 3 corresponding to the “zone C” (FIG. 4): Local transfer and storage of sanitized trolleys; phase 4 corresponding to the “zone D” (FIG. 5 and FIG. 6): Local removal of sanitized trolleys.

The first “zone A” corresponding, therefore, to the phase 1, analytically represented and described in the drawing 2 is the one arranged to receive the trolleys that the user puts after use. The trolley is placed through an automated door “3”, with mechanisms that provide safety protection for the user. Once the truck has entered the “zone A” the door “3” closes. At this point, in sequence, the door “5” is opened and the conveyor belt “4” is activated, actuated by means of sensors “31” placed below said belt, weight detectors which, in the presence of any objects forgotten inside the carriage, prevent the motor from being driven. The conveyor belt which acts as the floor of the “zone A” carries (axis y FIG. 2) the trolleys in the “zone B” of sanitization (phase 2), with the aid of a mechanical arm hooked to the ceiling described in greater detail below.

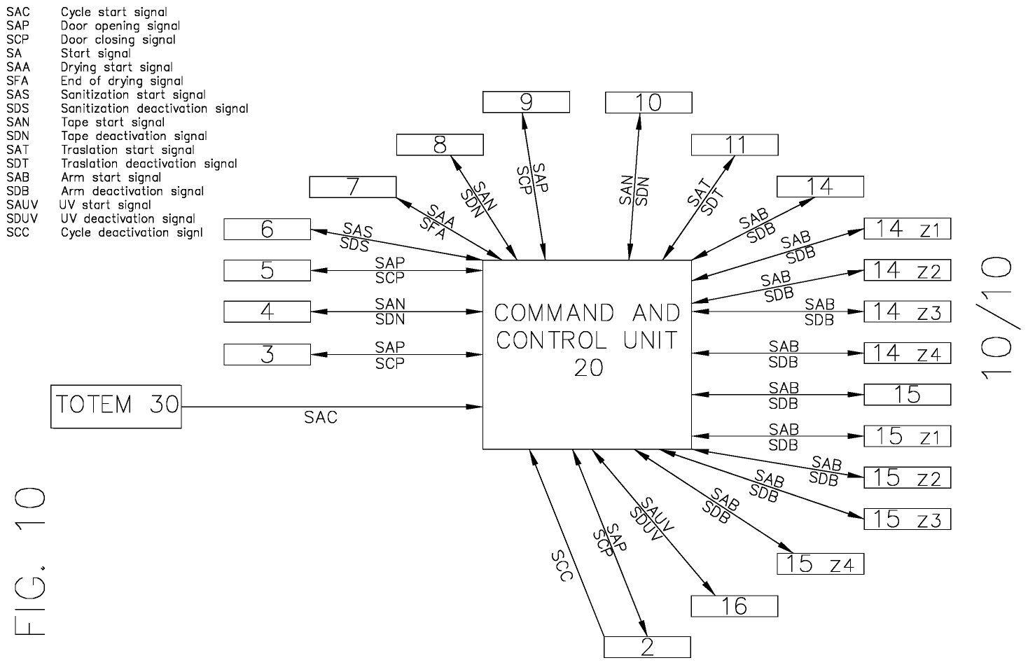

The “zone B” is intended to receive the carriages that access it through the door “5” which, after the passage, closes again (FIG. 3). At this point, in a programd sequence, the step of disinfecting and sanitizing the trolleys is started by using a sanitizing solution contained in a suitable tank “21” of the indicative capacity of lt. 300 (FIG. 3). This occurs through n. 4 or more nozzles “6” atomizing the disinfectant with 2 or more direction cannulas toward the carriages (both in the outer and inner part). The “zone B” is provided with underlying drawer “23” and liquid recovery tank “22” of result (FIG. 3), recovered through no. 1 pump that which sucks the waste liquid parts then collected in the above-mentioned special tank which, regularly, will be emptied from the outside. The periodic emptying cycle also provides for the discharge of the resulting substances. From the diagram of FIG. 3 it can be seen that, at the end of the sanitizing phase, drying systems are activated by means of n. 2 fans indicated in the drawing by “7”. At this point, the sanified and dried trolleys are intended for the subsequent step of displacement to deposit through the passage through a conveyor belt (“8” FIG. 3) from the door “9”, as shown in drawing 3. Both the door “5” and the door “9” of the “zone B” are hermetically sealed in such a way as to keep completely isolated the said zone “zone B” from the previous “zone A”, destined to the collection of the trolleys just used, which will remain isolated from the subsequent zones and, therefore, steps in order to avoid contamination between the carriages, thus ensuring the real sanitization thereof. Once the washing and drying step has been completed, the “zone C” comes into operation when the carriages are transferred onto the translation platform “11” (FIG. 4); as soon as the carriages have passed onto the platform, through the conveyor belt “10” installed therein, the door “9” is closed. Also in this phase of the plant it was considered appropriate to resort to a specific logic process with dedicated management software, by means of apparatus “20”, housed in the “zone A” (FIG. 2) that starts the displacement (axis y FIG. 4). and the location of the truck after identifying the first free storage lane. The translation platform, consisting of a conveyor belt mounted on a rail, can perform the horizontal translation (axis x FIG. 4), stop through sensors and, therefore, channel the carriages onto the parking lane. In this step, a mechanical arm “14” (phase C FIG. 4) is also activated, coupled to the ceiling of the plant which, moving on a suitable rail “29”, helps the positioning of the trolleys in the storage lane, automatically selected by the above software system. The lanes can be in the number of 2 and multiples (ZI . . . Zn FIG. 1), being the system designed according to modular criteria. In the final part, before the door “16” which opens to the “zone D” of the drawing 5, UV lamps “15” (FIG. 5) are operated, placed in such a way as to ensure a further disinfection of the handle of the trolley. This is in order to better guarantee and protect the user. In this last phase and “zone D” the user takes place. This area is also characterized by two separate doors “16” and “2” (FIG. 6). The hermetically sealed door “16”, like all the others, opens only for the automatic deposit in the “zone D” and, once the trolley has been deposited, closes automatically. With the opening of the door “16”, strictly closed and hermetically sealed, it is possible to allow the user, who lies in the area in front of the withdrawal (FIG. 6), to withdraw his sanified trolley and in perfect hygienic conditions. The opening of the said door and the subsequent automatic closure are subject to the payment according to the following procedure. Each lane of trolleys is provided with a pick-up zone aimed at ensuring social spacing and, for the same reasons, each pick-up zone is provided with a totem with an inner “30” box armoured for payment both in cash and in monetics, Either via APP with QR Code (FIG. 6). When the user has been served, when the door “02” is closed, the door “16” is automatically reopened to allow the repositioning in the “zone D” of another trolley already sanified and ready for the next withdrawal cycle. The system, in terms of the electromechanical operation, operates through a control and control unit (20), located in the above mentioned “zone A” (100) for opening and closing the doors, activating and deactivating the motors of the conveyor belts, for activating and deactivating the sanitizing and drying cycles, The activation and deactivation of the UV lamps, for the displacement and the positioning of the sanified carriages, configured to receive signals representative of cycle start-up—SAC—(FIG. 10) and to generate signals of duration and timing of start-up—SA—(FIG. 10), alternated with deactivation signals (SD).

It was believed to enrich the totem of the collection zone with Termocanner with temperature and mask presence detector. The realization of the plant, object of the present patent, allows to guarantee the almost total elimination of the viral and bacterial charges on the trolleys and will allow to considerably reduce the use of disposable gloves (currently used as dpi, but with doubts within the scientific community about their effective usefulness). and the subsequent phenomenon of their abandonment in an open place, of serious environmental impact and inevitably destined to amplify the already serious phenomenon of plastic pollution. Always with an eye attentive to the preservation and protection of the environment, the automated sanitizing plant produces no emission into the atmosphere, since the sanitizing procedure takes place in a completely closed and hermetically insulated environment, without any dispersion of chemical products and/or atmospheric emissions.

In practice, the details of construction and implementation may vary without departing from the scope of the invention and hence from the domain of the invention patent.

To enrich the plant in order to provide further services to the user and at the same time to make it even more attractive for the commercial activity that houses it, the installation of advertising panels and charge columns of electric cars has been provided. Specialists in the field, in order to satisfy contingent and specific requirements, may also make modifications and variations to the configurations described above. For example, the sanitizing liquid tank may be placed in a remote position with respect to the device, or the arrangement of the storage and removal zones C and D of the trolleys may vary or the number of dispensing nozzles which may be of any type.

These variants and modifications are all however contained within the scope of protection of the invention as defined by the related claims.

1. Fully automated system (1) for sanitizing shopping trolleys, consisting of 4 parts, identified as Zone A (100), Zone B (101), Zone C (102), Zone D (103) with sequential operation corresponding to 4 Phases, of which Zone A (100) used shopping trolley storage room, equipped with automated door (3), intended to house conveyor belt (4) and feed motors (20) for automation of conveyor belts (4) and for washing; said Zone A (100) also contains a general electrical panel (19) with inverter serving the off-grid photovoltaic system (24), system placed on the cover of said sanitizing apparatus (1), battery pack for power supply (18), totem (17), designed for the return of a partial amount—previously paid for the collection of the sanitized trolley—equipped with an internal armored cash box; “Zone B” (101) is hermetically separated from the said “Zone A” (100), by means of an automated door (5), which is designed to accommodate the trolleys to be sanitized, consisting of a shopping trolley sanitation room, which houses a sanitizing solution (21), liquid recovery tank (22), n. 4 dispensing nozzles (6), or more, designed to atomise the sanitizing solution, by means of 2 or more direction cannulas fed by a special pump, called “Zone B” (101), housing two fan towers (7) for the drying of the sanitized trolleys which, therefore, pass through the hermetically sealed automated door (9) to the next Zone C (102), distinct from the first (100) and the second (101), this is a storage room for shopping trolleys already perfectly sanitized, which houses a translation platform (11), equipped with sensors, consisting of a conveyor belt (10) mounted on a transverse rail (13) on which the trolleys are moved by means of said conveyor belt (10) and with the aid mechanical arm (14), hooked to the ceiling of the system which, moving on a special rail (29), helps the positioning of the trolleys in the free storage lane (Z), automatically selected by the software system, trolleys that are further disinfected titled by n. 2 UV lamps (15) for each lane (Z), placed before door 16 (16) which, when opened, gives access to “Zone D” (103), distinct and separate from the first (100), the second (101) and the third (102), set up for collection by means of an automated door 2 (2) which opens, after using a special totem (30), equipped with an internal armored cash box (25) which works both by cash payment (26) and by e-money (27), and by APP (28) with QR Code, designed to activate the sanitation cycle; said totem (30) is also equipped with a Termoscanner with temperature detector and the presence of a mask.

2. Totally automated system (1) for sanitizing shopping trolleys according to claim 1, in which said Zone A (100) consists of a storage compartment for used shopping trolleys, dosed by automated door “3” (3) in entrance and automated door “5” (5) for accessing the trolleys to the distinct Zone B (101), which opens when the aforementioned entrance door (3) is dosed, includes a conveyor belt (4) equipped with its own motor.

3. Totally automated system (1) for sanitizing shopping trolleys, according to claims 1 and 2, in which the conveyor belt (4) is activated by sensors, including weight detectors (31) placed under the belt itself (4).

4. Fully automated system (1) for sanitizing shopping trolleys, according to claims 1, 2 and 3 in which the said “Zone A” (100) includes totems (17), designed for the return of a partial amount—previously paid for withdrawal of the sanitized trolley—equipped with an armored internal cash box, as well as a general electrical panel (19) with inverter serving the off-grid photovoltaic system (24), with antenna/internet receiver (32) for remote management on the roof of the system (1), battery pack for power supply (18).

5. Fully automated system (1) for sanitizing shopping trolleys according to any one of the preceding claims, in which the said “Zone B” (101) consists of a compartment, which is accessed through the aforementioned automated door “5” (5), containing tank (21) of the indicative capacity of lt. 300, containing sanitizing solution which through n. four or more dispensing nozzles (6) is atomized, with two or more direction cannulas towards the trolleys, both internally and externally, with collection of the residual liquid parts of the sanitization process, falling in a cascade into the collection compartment (23) and recovered through n. 1 suction pump in a special liquid recovery tank (22).

6. Totally automated system (1) for sanitizing shopping trolleys according to any one of the preceding claims, in which said “Zone B” (101) comprises systems designed for drying the trolleys previously sprayed with the sanitizing solution, consisting of two towers with n. two fans (7).

7. Fully automated system (1) for sanitizing shopping trolleys according to any one of the preceding claims, comprising a command and control unit (20), located in the previous “Zone A” (100) for opening and dosing the doors, the activation and deactivation of the motors of the conveyor belts, for the activation and deactivation of the sanitizing and drying cycles, the activation and deactivation of the UV lamps for moving and positioning the sanitized trolleys, configured to receive signals representative of cycle start—SAC—(FIG. 10) and generate consequential and timed start signals—SA—(FIG. 10), interspersed with deactivation signals (SD).

8. Totally automated system (1) for sanitizing shopping trolleys according to any of the preceding claims, in which “Zone C” (102), distinct from the first (100) and the second (101), consists of the trolley storage room of the sanitized shopping, which houses a translation platform (11), equipped with sensors for activating and blocking the movement, consisting of a conveyor belt (10) mounted on a transverse rail (13) and which, through the rack (12), performs the translation horizontally, on the X axis.

9. Totally automated system (1) for sanitizing shopping trolleys according to any one of the preceding claims, in which said Zone C (102) is also equipped with a mechanical arm (14) hooked to the ceiling of the system which, moving on a special rail (29), helps the positioning of the trolleys in the free channeling and storage lane (Z), automatically selected by the software system by means of a control apparatus (20) housed in the said “Zone A” (100).

10. Totally automated system (1) for sanitizing shopping trolleys according to any one of the preceding claims, in which Zone C (102), distinct from the first (100) and the second (101), is equipped with n. 2 UV lamps (15), for each lane (z), placed before door “16” (16) which opens to Zone 4 and which come into operation for further disinfection and sanitization of the trolley handle.

11. Fully automated system (1) for sanitizing shopping trolleys according to any one of the preceding claims, in which “Zone D” (103), distinct from the first (100), the second (101) and the third (102), is consisting of a local picking up sanitized shopping trolleys, characterized by two distinct doors “16” (16) and “2” (2), called door “2” (2), when opened, allows the removal of the sanitized trolley ready for use. The opening of the said door “2” (2) is subject to the use by the user of the totem (30), located in each withdrawal area—equipped with an internal armored cash box (25) functioning either through cash payment (26), both in e-money (27) and via APP (28) with QR Code, set up for the activation of the sanitation cycle; said totem (30) is also equipped with a Termoscanner with temperature detector and the presence of a mask.
